(19) United States
(12) Patent Application Publication
(10) Pub. No.: US 2013/0317484 A1
(43) Pub. Date: Nov. 28, 2013

(54) METHOD OF LIPOSUCTION

(71) Applicants: Bruce Chau, Berkley, MI (US); Tony V. Lu, Bellevue, WA (US); Thomas Tsang, Brooklyn, NY (US)

(72) Inventors: Bruce Chau, Berkley, MI (US); Tony V. Lu, Bellevue, WA (US); Thomas Tsang, Brooklyn, NY (US)

(21) Appl. No.: 13/900,353

(22) Filed: May 22, 2013

Related U.S. Application Data
(60) Provisional application No. 61/650,096, filed on May 22, 2012.

(51) Int. Cl. A61M 1/00

(52) U.S. Cl. CPC A61M 1/008
USPC 604/542

(57) ABSTRACT

A method of liposuction that results in a smooth skin surface to a patient post procedure, by performing superficial liposuction on the patient by inching and fanning, obtaining an even skin flap, aspirating deep fat, and achieving a smooth contour of skin. SVF extracted from the method above.

### BACKGROUND OF THE INVENTION

1. Technical Field

The present invention relates to methods of liposuction. More specifically, the present invention relates to methods of reusing fat that has been extracted from a patient during liposuction.

2. Background Art

Liposuction is a cosmetic surgery operation that is the process of removing fat from beneath the skin through a cannula with a vacuum or aspiration source. Common areas of the body that liposuction is performed on include the abdomen, thighs, hips, buttocks, neck, and arms. Once the fat is removed from the body, it is generally discarded.

One of the drawbacks of current liposuction techniques is that they leave patients with palpable bumps or lumpiness in the abdomen for a prolonged period of time post procedure.

There remains a need for improvement of current liposuction techniques and the discarded fat from liposuction can be put to better use.

### SUMMARY OF THE INVENTION

The present invention provides for a method of liposuction that results in a smooth skin surface to a patient post procedure, by performing superficial liposuction on the patient by inching and fanning, obtaining an even skin flap, aspirating deep fat, and achieving a smooth contour of skin.

The present invention also provides for SVF extracted from the method above.

### DESCRIPTION OF THE DRAWINGS

Other advantages of the present invention are readily appreciated as the same becomes better understood by reference to the following detailed description when considered in connection with the accompanying drawings wherein:

FIG. 1 is a drawing of fat and a distal end;

FIG. 2 is a drawing of fat showing three zones;

FIGS. 3A-3C are drawings of aspirating fat from a distal zone, middle zone, and proximal zone;

FIG. 4 is a drawing of overlapping zones; and

FIG. 5 is a drawing of removing the superficial fat layer and the deep fat layer.

### DETAILED DESCRIPTION OF THE INVENTION

The present invention generally is directed to a method of liposuction that results in a smooth skin surface to a patient post procedure. The method includes the steps of performing superficial liposuction on the patient by inching and fanning, obtaining an even skin flap, aspirating deep fat, and achieving a smooth contour of skin.

The method is a safe and efficient technique in harvesting adipose tissue and leaves the donor site with minimal deformity. The concept of “Tumescent Infiltration” is well established. However, the manual technique used in the prior art is quite variable from surgeon to surgeon. The present method is a unique technique of liposuction. This technique is systematic, safe, teachable, and reproducible.

A superficial liposuction technique is the cornerstone for achieving a smooth contour after the procedure. Normally, once the deep fat has been aspirated, especially the deepest layer that is just above the muscle, the surgeon loses control in defining the evenness of the skin flap and the entire thickness of the skin. In addition, the fat has lost its anchoring support. Prior art methods have not performed superficial liposuction before deep fat liposuction. The superficial liposuction method of the present invention helps raise an even skin flap first with the advantage of the non-dominant hand of the surgeon acting as the guiding instrument. Superficial liposuction has added skin contraction benefits. Once the even and smooth skin flap is developed, the deep fat can be aspirated evenly and the smooth, even skin flap re-drapes the liposuction site to achieve a smooth contour.

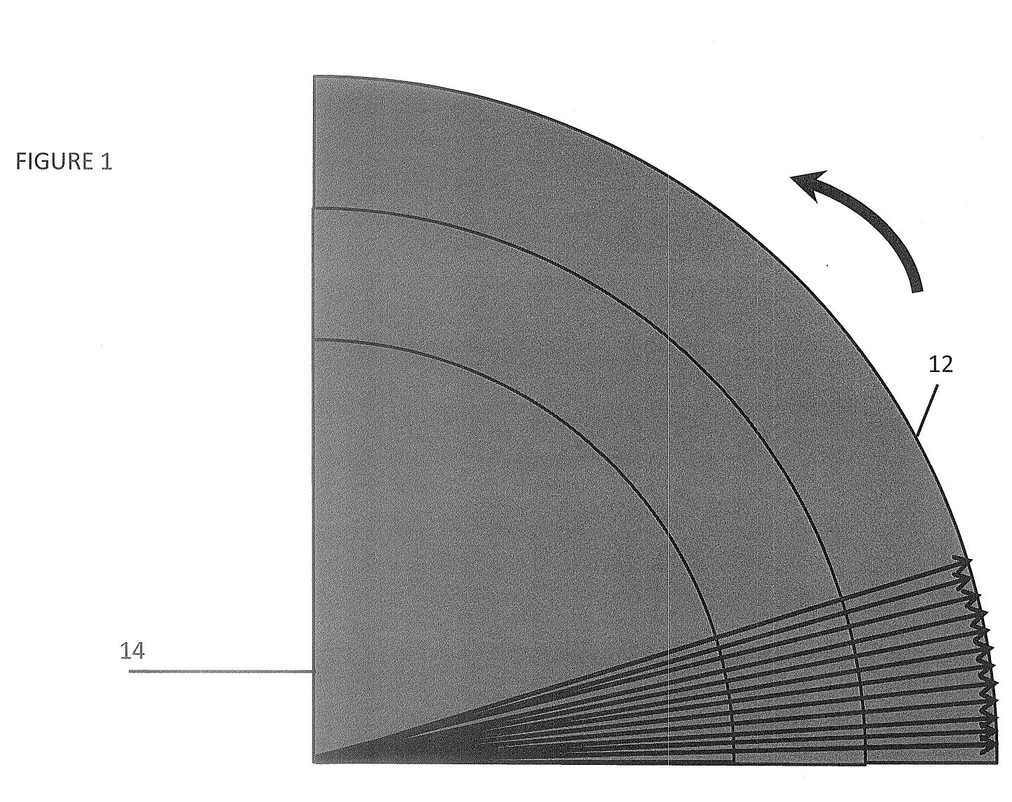

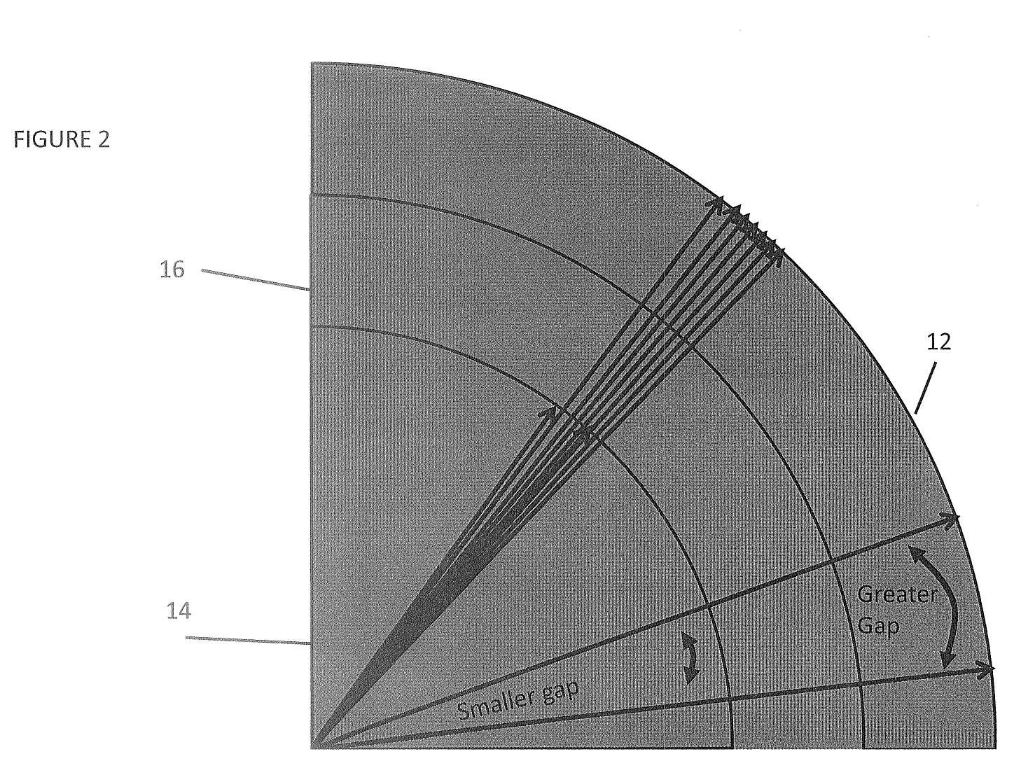

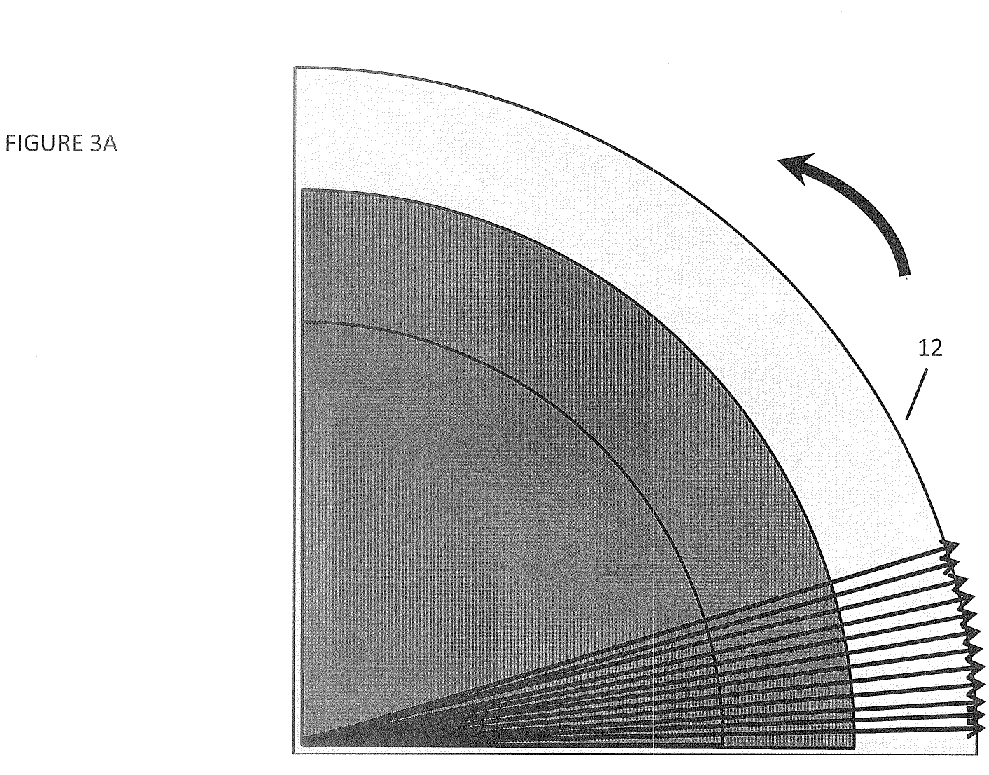

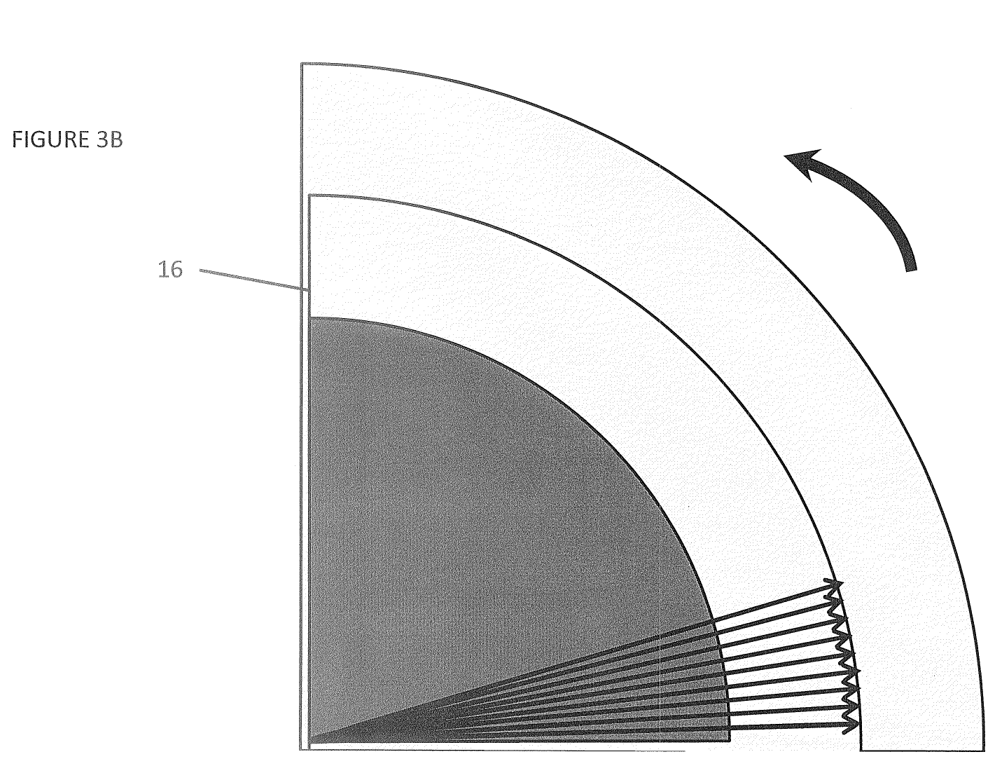

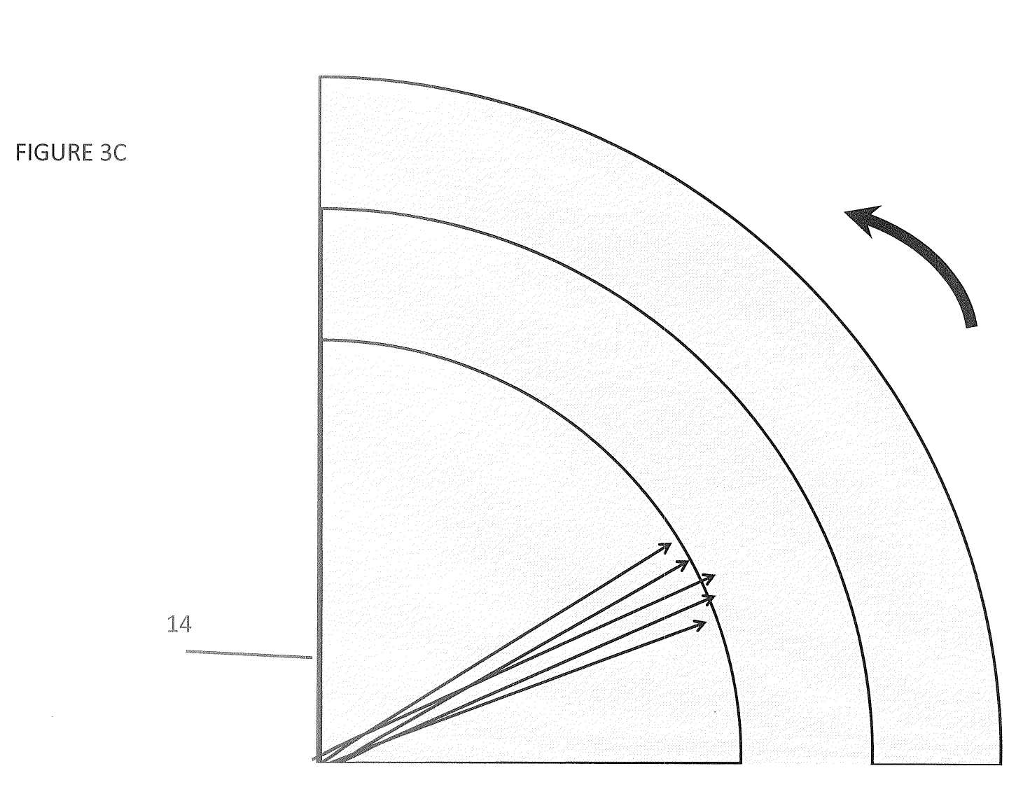

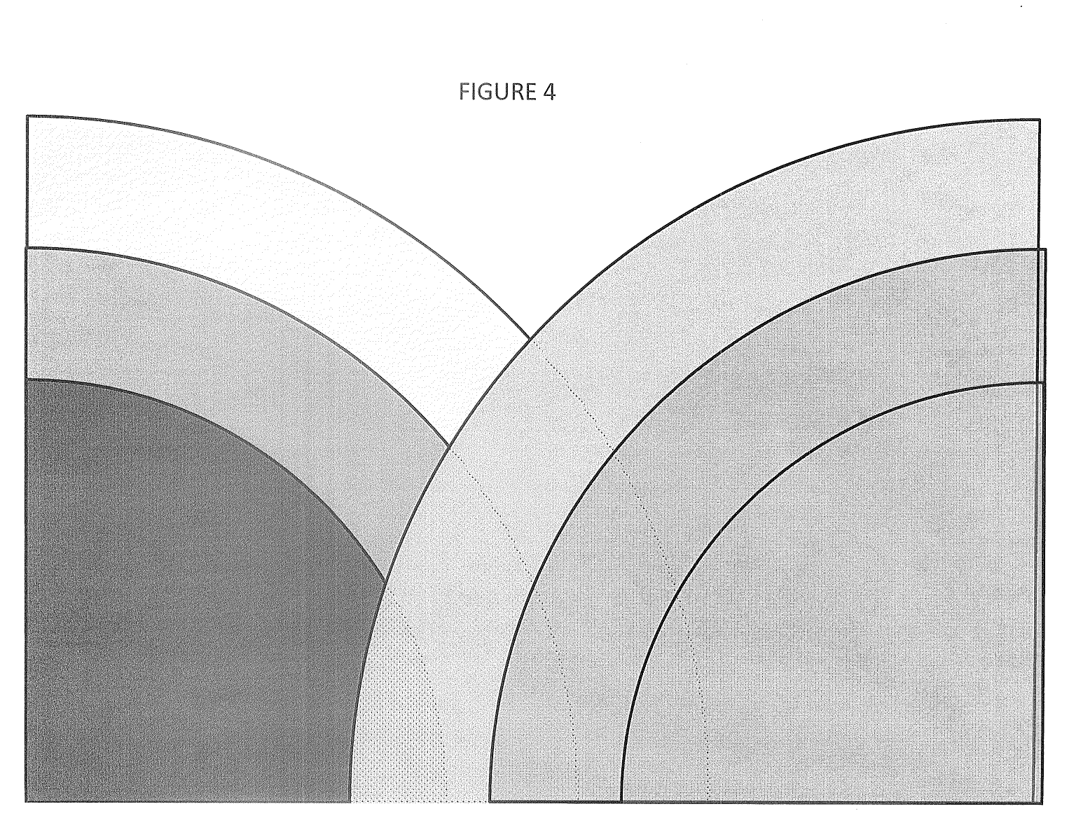

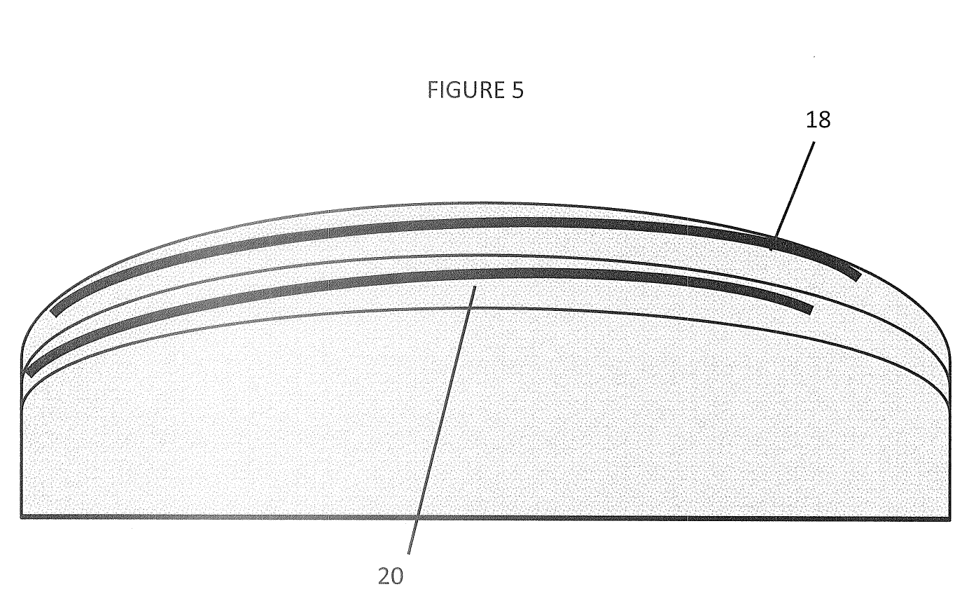

More specifically, the method involves preparation by tumescent infiltration and aspiration by harvesting fat graft. Standard tumescent fluid can be prepared in exemplary amounts and used in the method including lactate ringer (l liter), epinephrine (1:1000, 1 cc), sodium bicarbonate (8.4 mEq, 10 cc), and lidocaine (1%, 50 cc). An infiltrating cannula can be used (1.5 mm to 2.0 mm diameter, 15 cm to 25 cm in length). First, the distal zone 12 from the insertion site is infiltrated in the superficial fat layer. Infiltration progresses from left to right or from right to left in an “inching and fanning” manner. Prior art liposuction methods have never used inching and fanning. The inching is exactly adjacent to the last or prior cannula pass, as shown in FIG. 1. The distal zone 12 of the infiltration has a wider gap, as shown in FIG. 2. Therefore, more passes are needed to treat this area to fill the adipose tissue in between. It is advisable to infiltrate distally first because proximal fill-up can impede distal infiltration. The infiltration can be divided into three zones, a proximal zone 14, a middle zone 16, and the distal zone 12. Each zone can be infiltrated separately, as shown in FIGS. 3A-3C, going from distal 12, to middle 16, to proximal 14 at which point the infiltration in completed. Infiltration can be performed in an adjacent area so that overlapping of the zones occurs and this acts as pre-tunneling, as shown in FIG. 4. Pre-tunneling is passing the cannula back and forth without suction applied, and is performed similar to infiltration from right to left or left to right, except that the order of the zones is reversed. The proximal zone 14 is pre-tunneled first because the fat layer can be fibrous and it is imperative to loosen up the proximal zone in order to pave the way for the more distal areas. Pre-tunneling progresses further to the middle zone 16, and the distal zone 12. This criss-crossing and pre-tunneling occurs because there are gaps between each pass of the aspiration with a straight cannula. The superficial fat layer 18 is infiltrated first, and then the deep fat layer 20 is infiltrated and the cannula is directed to all layers to achieve a total 3D infiltration, as shown in FIG. 5.

A harvesting cannula can be used (2.0 mm to 2.7 mm, syringe suction or machine suction, and 15 cm to 25 cm length for a smooth contour larger area to re-drape skin). A sterile collecting canister with a valve to empty aspirated fluid or fat into syringes can be used.

In performing this technique, the surgeon must manipulate and flatten the tissue so that the cannula is parallel to the skin, which assumes a curve. This requires the flawless, concerted, and coordinated movements from both hands that are doing completely different tasks but complementary to each other. As the cannula advances, the surgeon feeds the fatty tissue toward the tip of the cannula and the tissue is spread to keep it parallel with the cannula. Meanwhile, the tactile sensation of the guiding hand ensures that the cannula stays in the same plane with the operating hand manipulating the cannula to the appropriate depth. An assistant can also maintain the skin parallel to the cannula. Since the cannula is straight, the skin is moved to keep it parallel. Once the desired amount of fat is harvested, the cannula is passed back and forth to ensure that an even and smooth surface is achieved. Further liposuction can be necessary in order to make sure the skin flap is smooth and even. The entry sites are sutured with 5-0 nylon or other appropriate sutures. The harvested fat is then spun down and treated to isolate SVF, described below. A smooth foam can be placed over the harvested area on the patient, and a compressive garment can be placed over the foam for comfort, control of swelling, and prevention of skin wrinkling. The patient is then taken to a recovery area and awaits the SVF preparation and infusion.

This method can be performed on any part of the body where excess fat needs to be removed, such as, but not limited to, the abdomen, arms, thighs, neck, back, chin, face, or combinations thereof.

The method of liposuction can be coupled with a method of extracting SVF (stromal vascular fraction) from fat (adipose) cells extracted from a liposuction procedure (such as the superficial liposuction described above) using the precise amount of collagenase to digest the fat cells and the ideal incubation time with an incubator with temperature and shaking motion control to achieve the highest number of cells in the SVF. The fat cells can be centrifuged with any appropriate centrifuge after treatment with collagenase to obtain the SVF (from the bottom portion of the centrifuge tube, a washing fluid portion and adipose portion are above the SVF in the centrifuge tube). The SVF can be transferred by sterile pipets for reinjection into the patient. This method is further detailed below in Example 1. The present invention provides for the SVF extracted by this method.

There are many purposes of extracting SVF. SVF is considered autologous (coming from your own body) and minimally manipulated before returning back to the same person. Therefore, the method of the present invention (Liposuction, SVF extraction and reinfusion) is compliant with the USFDA Code of Federal Regulations Title 21, part 1271 and fall under the same surgery exemption discussed in 1271.15(b). SVF contains mesenchymal stem cells, growth factors, pre-adipocytes, endothelial and pre-endothelial cells, stromal cells, and other non-cellular elements. As a result, SVF has the ability to repair damaged tissues and helps slow down the aging process. SVF extracted from discarded fat can provide additional healthcare and anti-aging options to the patients undergoing liposuction. SVF can be used in conjunction with other procedures such as face and hands rejuvenation, hair restoration, bone and joints injections, and sexual function enhancement to address total age management. SVF is safe and these innovative methods of utilizing SVF have seldom been offered to post surgical patients and the suctioned fat tends to be wasted and discarded.

The SVF can be extracted from the fat, activated, and returned to the patient intravenously for systemic rejuvenation or used in conjunction with other procedures to address total age management as stated above. SVF can be given intravenously and/or injected on the face, hands, scalp, into joints and sexual organ of the same individual. These methods of utilizing SVF have seldom been offered to post surgical patients and the suctioned fat tends to be wasted and discarded as described above. This method is further described in Example 2.

There are several advantages to the methods of the present invention. The smooth liposuction method overcomes the bumpiness problems following the procedure as in current liposuction methods. The SVF extraction technique puts unwanted fat to good use. SVF can help repair the degenerative process associated with aging. The methods of the present invention provide additional healthcare and anti-aging options to the patients undergoing liposuction surgery.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### EXAMPLE 1

### Fat Isolation Technique with Smooth Liposuction and SVF Extraction from Autologous Adipose Tissue

### Materials/Equipment

Lipoaspirate

Lactated Ringers or Phosphate Buffered Saline (PBS) or Hanks Balanced Salt Solution (HBSS)

Liberase TL

200-micron mesh strainer

40-micron filter

Centrifuge—swing out buckets for 15-50 mL tube

Centrifuge tube—50 mL

Erlenmeyer Filtering Flask—500 mL

Silicone Stopper

D5 Lactated Ringers or DMEM-glucose

### Method

LIPOASPIRATE WASHING

1. Place lipoaspirate into a 50 mL sterile Centrifuge tube

2. Centrifuge at 1000 rpm (g=140 for Dupont Sorvall rotor radius of 12.5 cm) for 2 minutes to separate the blood/fluid/oil from Adipose tissue.

3. Removed the blood/fluid/oil fraction with 60 mL syringe attach to 16 gauge spinal needle.

4. Add EQUAL volume of Lactated Ringers or PBS or HBSS

5. Shake for 10 Seconds

6. Centrifuge at 1000 rpm (g=140 for Dupont Sorvall rotor radius of 12.5 cm) for 2 minutes to separate the blood/fluid/oil from Adipose tissue.

7. Removed the blood/fluid/oil fraction with 60 mL syringe attach to 16 gauge spinal needle.

8. Repeat STEPS 4-7 until the final wash is CLEAR.

ENZYMATIC DIGESTION

1. Liberase TL—5 mg vial—reconstitute with 2 mL of Sterile Water

2. Place adipose tissue and collagenase in a 50 mL Centrifuge tube

From the experiments, the ideal concentrations of Liberase and incubation times are 1.25 ml/30 minutes and 1 ml/40 minutes for 24 ml of Lipoaspirate.

### Summary

The above method of liposuction coupled with SVF extraction from Lipoaspirate leads to a smooth abdominal surface post procedure and offers the opportunity to use the SVF for cellular repair, anti-aging, and regenerative purposes.

### EXAMPLE 2

### Process of SVF Extraction from Lipoaspirate

Wash the lipoaspirate content with Lactated Ringers solution until clear of blood product.

Use Liberase TL (a commercially available enzyme mixture) to digest and separate the SVF from the fat.

Wash SVF with Lactated Ringers solution three times.

Filter SVF through 40 micron cell strainer.

SVF intravenous infusion.

IV therapy with PRP, Calcium and Magnesium to activate the SVF.

### Summary

The above method is a safe technique to process the SVF extraction from Lipoaspirate, to activate and nourish the SVF, and to safely deliver the SVF back to the patient.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that the invention can be practiced otherwise than as specifically described.

What is claimed is:

1. A method of liposuction that results in a smooth skin surface to a patient post procedure, including the steps of:
performing superficial liposuction on the patient by inching and fanning;
obtaining an even skin flap;
aspirating deep fat; and
achieving a smooth contour of skin.

2. The method of claim 1, wherein said inching and fanning step is further defined as infiltrating a distal zone with a cannula and infiltrating in a manner chosen from the group consisting of right to left and left to right.

3. The method of claim 2, wherein each pass by the cannula is exactly adjacent to a prior pass.

4. The method of claim 2, wherein said inching and fanning step further includes infiltrating a middle zone and a proximal zone.

5. The method of claim 4, further including the step of pre-tunneling by pre-tunneling a proximal zone, pre-tunneling a middle zone, and pre-tunneling a distal zone.

6. The method of claim 1, wherein said obtaining step further includes a non-dominant hand of the surgeon acting as the guiding instrument, feeding fatty tissue towards a tip of the cannula, and spreading the tissue to be parallel with the cannula.

7. The method of claim 1, wherein said achieving step is further defined as the even skin flap re-draping the site of liposuction.

8. The method of claim 1, wherein said performing step is further defined as performing superficial liposuction on a site chosen from the group consisting of the abdomen, arms, thighs, neck, back, chin, face, and combinations thereof.

9. The method of claim 1, further including the step of extracting stromal vascular fraction (SVF) from fat cells.

10. The method of claim 9, wherein said extracting step further includes the steps of using a precise amount of collagenase to digest the fat cells and an incubation time to achieve the highest number of cells in the SVF.

11. The method of claim 10, further including the step of using the SVF in a procedure chosen from the group consisting of face and hands rejuvenation, hair restoration, bone and joints injections, and sexual function enhancement.

12. The method of claim 11, wherein the SVF is administered intravenously to the patient.

13. The method of claim 11, wherein the SVF is administered by injection to the patient.

14. The method of claim 11, wherein the SVF is administered to a site chosen from the group consisting of the face, hands, scalp, joints, and sexual organs.

15. The method of claim 11, further including the steps of the SVF repairing damaged tissues and slowing down the aging process.

16. The method of claim 11, further including the step of providing systemic rejuvenation.

17. Stromal vascular fraction extracted from the method of claim 9.
